(19) United States
(12) Patent Application Publication
(10) Pub. No.: US 2003/0109448 A1
(43) Pub. Date: Jun. 12, 2003

(54) Methods of promoting uptake and nuclear accumulation of polyamides in eukaryotic cells

(76) Inventors: Kathleen S. Crowley, Shrewsbury, MO (US); James K. Bashkin, University City, MO (US); Barbara A. Schweitzer, Webster Groves, MO (US); Dennis P. Phillion, St. Charles, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/289,677

(22) Filed: Nov. 7, 2002

Related U.S. Application Data
(60) Provisional application No. 60/419,753, filed on Oct. 18, 2002.
(60) Provisional application No. 60/348,148, filed on Nov. 7, 2001.

(51) Int. Cl. A61K 38/00; A61K 31/455

(52) U.S. Cl. 514/12; 514/355

(57) ABSTRACT

A method for enhancing cellular uptake and redistributing gene regulating polyamides from the extranuclear areas in eukaryotic cells to the nucleus thereof. The method consists of administering to the eukaryotic cells a molecular trafficking compound. The administered molecular trafficking compound affects cellular pathways and mechanisms that accumulate polyamides in cytoplasmic vessicles and/or efflux polyamides from the intracellular regions of cell. The method also includes modifying polyamides to contain negatively-charged or acidic moieties wherein the moieties inhibit the accumulation of polyamides in cellular lysosomes. By affecting the cellular pathways and mechanisms, the polyamides are redistributed throughout the cell, whereby nuclear accumulation of the polyamides is enhanced.

### CROSS-REFERENCE TO RELATED APPLICATION

This application claims priority from U.S. application Serial No. 60/419,753, filed Oct. 18, 2002 and U.S. application Serial No. 60/348,148, filed Nov. 7, 2001.

### FIELD OF THE INVENTION

The present invention is directed to methods of enhancing the uptake and nuclear localization of polyamides in a eukaryotic cell. The invention relates to modifying polyamide compounds in a manner which enhances uptake, reduces or inhibits the efflux of polyamides, and causes the distribution of polyamides within the cell, preferably resulting in an accumulation of polyamides in the nucleus. The invention also relates to compounds, compositions, and methods of administration thereof which enhance uptake of polyamides, reduce or inhibit the efflux of polyamides, and cause the distribution of polyamides within the cell, preferably resulting in an accumulation of polyamides in the nucleus.

### BACKGROUND OF THE INVENTION

Biotechnical research has recently discovered that polyamide compounds, in particular, oligomers comprising pyrrole (N-methyl pyrrole or “Py”) and imidazole (N-methyl imidazole or “Im”) ring structures, can be used to bind to the double stranded DNA of a cell. Additionally, other compounds, such as beta-analine and 3-hydroxypyrrole (“Hp”) can be included in the oligomers to further selectively bind to DNA base pairs. See FIG. 1. Dervan, Curr. Opin. Chem. Biol., 688, (1999).

The side-by-side pairing of the pyrrole and imidazole ring structures selectively bind to base pairs in the DNA minor groove. A pyrrole opposite a pyrrole (Py/Py) selectively binds to an A/T, T/A base pair. An imidazole opposite a pyrrole (Im/Py) selectively binds to a G/C base pair, while Py/Im selectively binds to a C/G base pair. Hp/Py selectively prefers binding to a T/A base pair and conversely a Py/Hp pair selectively binds to an A/T base pair. Beta-aniline may be opposite either another beta-analine or a Py to selectively bind to an A/T, T/A base pair. See Dickenson, L. A. et al., Journal of Biological Chemistry, Vol. 274, 12765-12773, (1999).

Polyamide compounds may also include gamma-aminobutyric acid (“γ”) in order to form a hairpin polyamide compound. Such a structure has been found to significantly increase the binding affinity of the polyamide to a target sequence of DNA.

Baird et al. describe solid phase synthesis of polyamides wherein the polyamides contain imidazole and pyrrole amino acids, gamma-aminobutyric acid (“γ”), as well as β-analine (“β”). In Baird's solid phase synthesis of the polyamides, the polyamide is cleaved from the solid support by aminolysis with N,N-dimethylamino)propylamine (“Dp”). See Baird, E. E. et al., J. Am. Chem. Soc. Vol. 118, No. 26, 6141-6146, (1996).

The binding of polyamide compounds to DNA has created a variety of potential benefits, including diagnostics and manipulation of gene expression. Thus far, polyamide research has been typically conducted in vitro on bacterial prokaryotic cells and lower level eukaryotic cells such as yeast cells wherein the polyamides enter the cell and bind to the cellular DNA.

Difficulties have been encountered, however, when research is conducted on higher level eukaryotic cells such as mammalian or other higher level animal cells. In order for polyamides to be effective in higher level eukaryotic cells, they must enter the cell, migrate through the cytoplasm, cross the nuclear membrane, and accumulate in the nucleus wherein they can bind to the DNA. While polyamides have been successfully bound to bacterial DNA and yeast DNA, experiments on higher level eukaryotic cells indicate that polyamides accumulate in cytoplasmic vesicles, lysosomes, or other vesicles in the cytoplasm. The vesicles subsequently efflux the polyamides from the cells, reducing the polyamide concentration within the cell thereby reducing the accumulation of polyamides in the nucleus where they bind to the DNA.

In order for polyamides to be effectively utilized in higher level eukaryotic cells, a beneficial method is needed wherein polyamides can be taken up by a target cell and accumulate in the nucleus wherein they can bind to cellular DNA.

### SUMMARY OF THE INVENTION

Among the various aspects of the present invention, therefore, is the provision of a method to enhance the uptake of polyamides within a eukaryotic cell, the provision of a method to reduce or inhibit the efflux of polyamides within a eukaryotic cell, the provision of a method to distribute polyamides within a eukaryotic cell, the provision of a method of gene regulation treatment wherein one or more polyamides are administered with a molecular trafficking chemical that enhances the accumulation of the polyamides in the nucleus of a eukaryotic cell, and the provision of a method to modify cellular proteins, pathways, or mechanisms of action that results in a distribution of a polyamide agent to the nucleus within eukaryotic cells.

Briefly, therefore, the present invention is directed to a method for modulating the distribution of a polyamide within eukaryotic cells. The method comprises administering a polyamide and a molecular trafficking compound to eukaryotic cells. The molecular trafficking compound is selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof.

The present invention is further directed to a method for modulating the distribution of a polyamide within eukaryotic cells wherein the polyamide is modified to contain an acidic moiety and the modified polyamide is administered to eukaryotic cells.

The present invention is further directed to a composition for modulating the expression of a gene in a eukaryotic cell. The composition comprises a polyamide and a molecular trafficking compound wherein the molecular trafficking compound is selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof.

Other objects and features of this invention will be in part apparent and in part pointed out hereinafter.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic of (N-methyl pyrrole) (Py), N-methyl imidazole (Im), and 3-hydroxypyrrole (Hp) ring structures.

FIG. 2 is a schematic of (N,N-dimethylamino)propylamine and (N-methyl amino)di-propylamine polyamide amine tail structures.

FIG. 3 is a schematic of Im-Py-Py-Py-γ-Py-Py-Py-Py-β-Ta-BODIPY-FL-X (Compound 1) and Im-Im-Py-Py-γ-Py-Py-Py-Py-β-Ta-FITC (Compound 2) structures.

FIGS. 4A and 4B are is a photographic images of RSF cells cultured overnight at 37° C. without 1 μM BIODIPy-labeled polyamide (Compound 1) and counterstained with DAPI just prior to imaging.

FIGS. 4C and 4D are photographic images of RSF cells cultured overnight at 37° C. with 1 μM BIODIPy-labeled polyamide (Compound 1) and counterstained with DAPI just prior to imaging. Green fluorescence from Compound 1 is observable with a fluorescein filter set within RSF cells (FIG. 4C). With a DAPI filter set, the green fluorescence from Compound 1 is excluded from the DAPI-stained (blue fluorescence) nuclei (FIG. 4D), but not in untreated cells (FIG. 4A).

FIGS. 4E and 4F are photographic images of HCT116 cells cultured overnight at 37° C. with 1 μM BIODIPy-labeled polyamide (Compound 1) and counterstained with DAPI just prior to imaging. Green fluorescence from Compound 1 is observable with a fluorescein filter set within HCT116 cells (FIG. 4E). With a DAPI filter set, the green fluorescence from Compound 1 is excluded from the DAPI-stained (blue fluorescence) nuclei (FIG. 4F), but not in untreated cells (FIG. 4A).

FIG. 5 are images illustrating colocalization of BODIPY-labeled polyamide with organelle-specific fluorescent probes in HCT116 cells. Cells were cultured overnight at 37° C. with 1 μM BIODIPY-labeled polyamide (Compound 1) (A, D, G) and counterstained with MITOTRACKER Red CM-H₂Xros (B), LYSOTRACKER Red DND-99 (E) or BODIPY TR ceramide (H). Overlayed fluorescence images of the same field captured using the fluorescein filter and the rhodamine filter set (C, F, I).

FIG. 6 are images illustrating the affect of verapimil in the localization of polyamides in the nuclei of synovial fibroblasts. Cells were cultured overnight at 37° C. with 1 μM BIODIPY-labeled polyamide (Compound 1) in the presence or absence of 100 μM verapamil and counterstained with DAPI just prior to imaging. With a fluorescein filter set, green fluorescence from Compound 1 is (A) observed within RSF cells in the cytoplasm in the absence of verapamil and (B) observed in the nuclei in the presence of verapamil.

FIG. 7 are images illustrating the nuclear localization of fluorescein-labeled polyamides in HCT116 cells. Cells werecultured overnight at 37° C. with fluorescein-labeled polyamide (Compound 2). The green fluorescence from Compound 2 is observed in the nuclei of the cells (B) when imaged using the fluorescein filter. Compound 2 is also observed in the nuclei of the cells (C) in an overlay of (A) and (B).

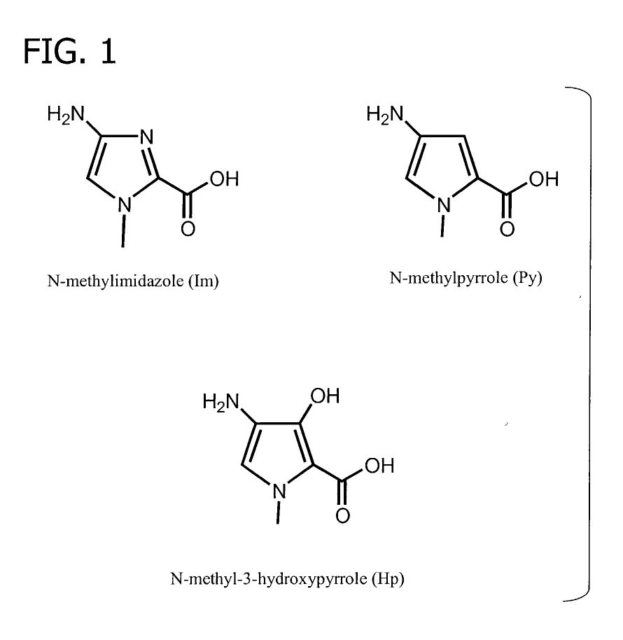

### DETAILED DESCRIPTION OF THE PREFERRED EMBODIMENTS

Naturally occurring pyrrole-containing polyamides such as distamycin and netropsin bind with high affinity to the minor groove of DNA. Since polyamides have been shown to interfere with protein-DNA interactions, they may also be utilized for regulating gene expression within cells. Synthetic polyamides can also be designed with sequence recognition capability to bind in the minor groove of DNA. For polyamides to be effective in gene regulation, however, they must reach target DNA inside the nucleus of the cell. In eukaryotic cells, polyamides must not only cross the plasma membrane, but they must also pass through the cytoplasm and cross the nuclear envelope to bind to the target DNA.

The present invention relates to methods of modulating the cellular uptake and distribution of polyamide compounds within a eukaryotic cell. More particularly, the present invention relates to enhancing the cellular uptake, reducing or inhibiting the efflux of polyamides, and promoting or enhancing the accumulation of polyamides in the nucleus of eukaryotic cells. Once in the nucleus, the polyamides can bind to target DNA in order to act as gene regulators.

Some cells exhibit a resistance to a variety of chemicals that lack a structural similarity and which may have different molecular targets. The resistance to chemicals such as polyamides may be exhibited through multiple pathways and is often related to enhanced efflux of the chemicals from the cell. Generally, the efflux process occurs with the accumulation of weakly basic compounds such as polyamides in acidic or lysosomal vesicles, the transport or migration of the vesicles to the plasma membrane, and the efflux of the polyamides from the cell. This resistance may be manifested by a variety of pathways and mechanisms described below or by one or more pathways or mechanisms of action that are unknown.

A variety of mechanisms or pathways may be utilized by eukaryotic cells to control the presence and movement of chemicals within a cell. In accordance with the present invention, it has surprisingly been discovered that chemical compounds that influence the movement or molecular trafficking of chemicals (hereinafter collectively referred to as “molecular trafficking compounds” in eukaryotic cells may be used to influence the distribution of polyamides into the nucleus of a eukaryotic cell. Such molecular trafficking compounds may promote the nuclear accumulation of polyamides through a variety of pathways, such as enhancing the cellular uptake of polyamides into the cell, inhibiting the efflux of polyamides from the cell, inhibiting vesicular sequestration or accumulation of polyamides, and/or affecting other intracellular molecular trafficking pathways and mechanisms.

Some eukaryotic cells possess proteins which act as molecular pumps to move chemicals through the cell as well as control the influx and efflux of the chemicals across the cell membrane. P-glycoprotein (P-gp), multidrug resistance protein (MRP), and canalicular multi-specific organic anion transporter (c-MOAT) are three such proteins that belong to the ATP-binding cassette (ABC) superfamily of transporters. ABC transporter proteins encompass a large family of over 200 known prokaryotic and eukaryotic transporter proteins. These proteins are believed to influence the concentration of chemicals within a cell by being associated with the mechanism wherein chemicals are effluxed from the cell through the cell membrane. The ABC protein pumps obtain the energy required for this function from ATP.

The ABC family of transporter proteins may also indirectly reduce the cellular concentration of chemicals by affecting the sequestration of chemicals in cytoplasmic vesicles by creating a pH or proton gradient across the vesicle membrane. The presence of a pH gradient is thought to promote the accumulation of basic chemicals into cytoplasmic vesicles and lysosomes. The lysosomes, in turn, migrate to the plasma membrane and subsequently efflux their contents to the extracellular region. Thus, while a polyamide may itself not be a substrate for an ABC transport protein, the protein is required to establish the pH gradient that causes the accumulation of polyamides in the vesicles. These mechanisms result in the removal of polyamides from intracellular cytoplasm and their reduction in their concentration within a cell.

Some molecular trafficking compounds can affect the normal function of ABC transporter proteins in a number of different pathways or mechanisms. Some of the mechanisms include direct binding of chemicals to the ABC proteins, chemically depleting ATP, inhibiting or enhancing the function of ATPase, blocking the efflux of vesicles, disrupting vesicular pH gradients, and the like.

Molecular trafficking compounds that affect pathways through direct or indirect inhibition of the ABC proteins may thereby enhance the cellular uptake of polyamides as well as reduce or inhibit the removal of polyamides from the cell. Thus, some molecular trafficking compounds that bind directly to the ABC proteins may inhibit their ability to function as efflux pumps by preventing the proteins from binding directly to the polyamides.

Other molecular trafficking compounds may reduce or inhibit the ability of the proteins to establish a pH gradient across cellular or vesicle membranes by cutting off their source of energy derived from ATP. Some molecular trafficking compounds act by directly depleting the intracellular ATP present in the cytoplasm. Other molecular trafficking compounds affect the availability of ATP energy by enhancing or inhibiting the activity of cellular ATPase or ABC protein-bound ATPase. By depleting or reducing the availability of ATP derived energy, the ABC transporter proteins are unable to establish a pH gradient in which polyamides are accumulated in vesicles. If a molecular trafficking compound prevents the accumulation of polyamides in vesicles, the concentration of polyamides in the cytoplasm will increase. As the concentration of polyamides increases in the cytoplasm, the resulting concentration gradient created between the cytoplasm and the nucleus causes the polyamides to migrate from the cytoplasm into the nucleus of the cell where they can subsequently bind to the cell's DNA.

Other molecular trafficking compounds may increase the concentration of polyamides in the cell by acting as calcium channel blockers within the cell which block the efflux of lysosomes. As more polyamides enter the cell and the cell fills with lysosomes which cannot efflux their contents in the extracellular regions, the ability of the cell to accumulate polyamides in lysosomes becomes overwhelmed. This results in an increased concentration of polyamides in the cytoplasm, which in turn causes the polyamides to migrate into the nucleus where they bind to the cell's DNA.

Still other molecular trafficking compounds act as chlorine channel blockers. Such molecular trafficking compounds disrupt the accumulation of polyamides within the lysosome through affecting the pH or proton gradient across the lysosome membrane. By preventing the polyamide accumulation within the lysosomes, the diffusion of polyamides throughout the cell is promoted resulting in the accumulation of polyamides in the nucleus.

Additionally, a large number of molecular trafficking compounds exist that redistribute polyamides from the extranuclear region within a cell to its nucleus through pathways or mechanisms of action in which one or more pathway or mechanism of action is unknown. These chemicals are generally identified in the art as multidrug resistance agents, modulators, compounds, and the like. These compounds shall be collectively referred to herein as “Multidrug Resistance Compounds.”

A number of molecular trafficking compounds may be used to affect the cellular uptake, reduced or inhibited efflux, and distribution of polyamides within a cell to the cell's nucleus. Examples of these molecular trafficking compounds are provided in Tables 1 to 9. Many of these compounds, as they may simultaneously affect a number of intracellular pathways, may be listed in more than one table. In addition to the chemicals listed in Tables 1 to 9, one skilled in the art would understand that derivatives of the chemicals (e.g., esters, acylated derivatives, and salts thereof) could elicit a similar cellular response.

A number of molecular trafficking compounds may be administered that affect or inhibit the normal functions of the P-gp ABC transporter protein. Examples of these chemicals are provided in Table 1.

TABLE 1
P-Glycoprotein Inhibitors

| | Reference to be |
|---|---|
| | Incorporated Herein |
| Molecular trafficking compound | in its Entirety |
| abamectin | |
| acridonecarboxamides | |
| aldosterone | |
| anthranilic acids of formula (I), (Ia), | U.S. 6,218,393 |
| (A), (B), (C), and (D) | |
| bepridil | |
| bepridil | |
| captropril | |
| clomiphene | |
| cortisol | |
| cyclosporin A | |
| cyclosporin D | |
| cyclosporin F | |
| dexamethasone | |
| diarylalkyl piperidines | U.S. 5,648,365 |
| dihydropyridine | |
| diltiazem | |
| dipyridamole | |
| doramectin | |
| emetine | |
| eprinomectin | |
| essential oils | U.S. 6,121,234 |
| estramustine | |
| FK-506 | |
| Formula I Compounds | U.S. 6,297,216 |
| Formula I, II, III, and IV Compounds | U.S. 5,726,184 |
| Formula Ia and IIa Compounds | U.S. 6,248,752 |
| hydroxychloroquine | |
| ivermectin | |
| liposomes | |
| macrocylic lactone compounds | U.S. 6,114,376 |
| megestrol acetate | |
| milbemycin A | |
| milbemycin D | |
| NDGA or Analogs thereof | U.S. 5,541,232 |
| nifedipine | |
| phenothiazines | U.S. 6,245,805 |
| phenothiazines | |
| phenylpiperidine | |
| prednisone | |
| progesterone | |
| quercetin | |
| quinacrine | |
| quinidine | |
| quinine | |
| reserpine | |
| staurosporin derivatives of formula (I) | U.S. 5,827,846 |
| staurosporine | U.S 5,827,846 |
| tamoxifen | |
| terfenadine | |
| thioxanthenes | |
| trifluoroperazine | |
| Tumor Necrosis Factor | |
| verapamil | |
| vindoline | |
| vitamin A | |

Molecular trafficking compounds may be administered which affect the ATPase enzymes in the cell in a manner which promotes or enhances the distribution of polyamides to the nucleus. ATPase affecting molecular trafficking compounds may either enhance or inhibit cellular ATPase or P-gp bound ATPase. Molecular trafficking compounds that inhibit other ATPase present in the cell, such as mitochondrial inhibitors, can deprive the cell of energy derived from ATP, disrupt normal cellular efflux pathways, and result in the accumulation of polyamides in the nucleus. Molecular trafficking compounds that enhance P-gp bound ATPase can inhibit the P-gp's transporter function or the P-gp's ability to establish a pH gradient in lysosomes. This likewise results in enhanced accumulation of polyamides in the nucleus. Examples of ATPase affecting molecular trafficking compounds are provided in Table 2.

TABLE 2
ATPase Affecting Molecular trafficking compounds

| | Reference to be |
|---|---|
| | Incorporated Herein |
| Molecular trafficking compound | in its Entirety |
| alkaline phosphatase inhibitors | U.S. 6,121,234 |
| bafilomycin | |
| farnesyl-glutamyl-cysteine methyl ester | U.S. 5,571,687 |
| N-acetyl-S-Farnesylcysteine methyl amide | U.S. 5,571,687 |
| (AFCMA) | |
| N-acetyl-S-Farnesylcysteine methyl ester | U.S. 5,571,687 |
| (AFCME) | |
| oligomycin | |
| ouabain | |
| peat active factors | U.S. 6,267,962 |
| prenylcysteine compounds | U.S. 5,571,687 |
| reserpine | |
| S-farnesylcysteine methyl amide (FCMA) | U.S. 5,571,687 |
| S-farnesylcysteine methyl ester (FCME) | U.S. 5,571,687 |
| S-geranylgeranylcysteine methyl ester | U.S. 5,571,687 |
| (GGCME) | |
| tamoxifen | |
| tetrabenazine | |
| vanadate | |

Other molecular trafficking compounds have been found to affect the pH or proton gradients required for vesicular transportation. By disrupting the vesicular acidification, such molecular trafficking compounds can inhibit the uptake of chemicals from the cytoplasm and into acidic vesicles. Thus, pH or proton gradient disrupters can be administered to reduce the accumulation of polyamides in cytoplasmic vesicles. Examples of pH or proton gradient disrupter molecular trafficking compounds are provided in Table 3.

TABLE 3
pH or Proton Gradient Disrupters
Molecular trafficking compound

ammonium chloride
carbonyl cyanide m-chlorophenylhydrazone
chloroquine
2,4-dinitrophenol
esipramine
reserpine
tamoxifen
tetrabenazine
p-trifluoromethoxyphenylhydrasone (FCCP)
verapamil

Other molecular trafficking compounds have been found to be calcium channel blockers that inhibit the ability of lysosomes to efflux their contents. By inhibiting the lysosomal efflux of polyamides, the concentration of polyamides contained in the cell doesn't decrease and results in a polyamide concentration gradient within the cell that favors the movement of polyamides into the nucleus. Calcium channel blockers also act to inhibit P-gp. Examples of calcium channel blocking molecular trafficking compounds are provided in Table 4.

TABLE 4
Calcium Channel Blockers

| | Reference to be |
|---|---|
| | Incorporated Herein |
| Molecular trafficking compound | in its Entirety |
| amlodipine | |
| bepridil | |
| calmodulin inhibitors | U.S. Pat. Nos. |
| | 6,087,370, 5,670,507 |
| cyclosporin A | |
| diltiazem | |
| felodipine | |
| FK506 | |
| flunarizine | |
| isradipine | |
| nicardipidine | |
| nifedipine | |
| nimodipine | |
| nisoldipine | |
| nitrendipine | |
| quinine | |
| quinidine | |
| rapamycin | |
| reserpine | |
| tetrabenazine | |
| tiapamil | |
| trifluoperazine | |
| verapamil | |

Other molecular trafficking compounds have been found to be intracellular depleters of ATP. Such compounds deplete the cell of ATP, thereby suppressing P-gp activity. Examples of ATP depleting molecular trafficking compounds are provided in Table 5.

TABLE 5
ATP Depleting Molecular trafficking compounds
Molecular trafficking compound

L-alanosine
antimycin
azide and salts and derivatives thereof
2-deoxyyglucose and salts and derivatives thereof
glucono-delta-lactone
oligomycin and salts and derivatives thereof
valinomycin and salts and derivatives thereof

Some molecular trafficking compounds block the sodium and potassium ion channels within the cell and disrupts the pH balance within the cell. An example of sodium/potassium blocking molecular trafficking compounds is Quinidine.

Multidrug resistance proteins (MRP), like P-gp, act as transporters that remove chemicals from the cell. Thus, like inhibitors of P-gp, molecular trafficking compounds that inhibit the normal function of MRP can contribute to the distribution of polyamides to the nucleus. Examples of MRP inhibiting molecular trafficking compounds are provided in Table 6.

TABLE 6
MRP Inhibitors

| | Reference to be |
|---|---|
| | Incorporated Herein |
| Molecular trafficking compound | in its Entirety |
| acridonecarboxamides | |
| bepridil | |
| Dendroamine derivatives of Compound A | U.S. 5,869,650 |
| estramustine | |
| Formula I, II, III, and IV Compounds | U.S. 5,726,184 |
| Formula Ia and IIa Compounds | U.S. 6,248,752 |
| genestein | |
| megestrol acetate | |
| phenothiazines | |
| sodium orthovanadate | |
| thioxanthenes | |
| verapamil | |

Other molecular trafficking compounds have been found to inhibit protein kinase. Protein kinase induces the expression of the multidrug resistance genes which encode P-gp and MRP. Thus, by inhibiting protein kinases, P-gp and MRP will not be produced. Examples of protein kinase inhibitors are provided in Table 7.

TABLE 7
Protein Kinase Inhibitors

| | Reference to be |
|---|---|
| | Incorporated Herein |
| Molecular trafficking compound | in its Entirety |
| calphostin C | |
| chelerythrine | |
| D,L-threosphingosine | |
| erbstatin and its analogues | |
| genistein | |
| H7 | U.S. 5,972,598 |
| herbimycin A | |
| methyl-1,5-dihydroxycinnamate | |
| neomycin sulfate | |
| staurosporine | |
| suramin | |
| tyrphostin A25 | |
| tyrphostin B46 | |

A number of molecular trafficking compounds generally promote or enhance the distribution of polyamides in the nucleus through one or more pathway or mechanism wherein the pathway or mechanism may be unknown. Examples of these molecular trafficking compounds are provided in Table 8.

TABLE 8
Multidrug Resistance Compounds

| | Reference to be |
|---|---|
| | Incorporated Herein |
| Molecular trafficking compound | in its Entirety |
| acridonecarboxamides | |
| amiodarone | |
| amitriptyline | |
| bepridil | |
| berbamine | |
| biperiden | |
| cephalosporins | |
| cepharanthine | |
| chloroquine | |
| chlorpromasine | |
| cinchonidine | |
| cinchonine | |
| cyclosporin A | |
| demethoxyverapamil | |
| dendroamine derivatives of Compound A | U.S. 5,869,650 |
| dendroamine derivatives of Compound A | U.S. 5,869,650 |
| dihydrocinchonine | |
| diltiazeme | |
| d-Tetrandrine | |
| estramustine | |
| ethyl fangchinoline | |
| fangchinoline | |
| Formula (A) Compounds | U.S. 6,180,633 |
| Formula (C) compounds | U.S. 5,776,939 |
| Formula A Compounds | U.S. 6,130,219 |
| Formula I and II Compounds | U.S. 5,744,485 |
| Formula I and II Compounds | U.S. 5,935,954 |
| Formula I and II Compounds | U.S. 5,620,971 |
| Formula I Compounds | U.S. 5,723,459 |
| Formula I, II, III, and IV Compounds | U.S. 5,717,092 |
| Formula I, II, III, and XXXI Compounds | U.S. 5,543,423 |
| Formula Ia and IIa Compounds | U.S. 6,248,752 |
| hernandezine | |
| hydrocortizone | |
| hydroquinidine | |
| isotetrandrine | |
| lidocaine | |
| long chain amino alcohols of formula I | U.S. 5,670,507 |
| megestrol acetate | |
| N-substituted-1,5-didcoxy-1,5-imino-D- | U.S. 6,225,325 |
| glucitol, or galactitol compound, or | |
| pharmaceutically acceptable salt thereof | |
| of Formula I | |
| organoselenones (R₁—Se(O₃)—(CH₂)ₙ—X) | U.S. 5,614,562 |
| pentazocine | |
| phenothiazines | |
| phthalazinone derivatives of formulas I, | U.S. 5,556,856 |
| II, and III | |
| piperazine derivatives of formulas (A), | U.S. 5,852,018 |
| (Aa), (Ab), and (B) | |
| piperazinedione compounds of Formula (I) | U.S. 5,935,955 |
| potassium canrenoate | |
| progesterone | |
| progesterone derivatives of formula I | U.S. 6,143,737 |
| promethazine | |
| propanolol | |
| quinidine | |
| quinine | |
| reserpine | |
| salbutamol | |
| sdbethylene diamine | |
| staurosporin derivatives of formula (I) | U.S. 5,827,846 |
| tamoxifen | |
| thioridazine | |
| thioxanthenes | |
| toremifen | |
| trifluoperazine | |
| verapamil | |

In one embodiment, the present invention may be used as a method of modulating the distribution of polyamides within eukaryotic cells. More preferably, molecular trafficking compounds may be used to modulate the distribution of polyamides within eukaryotic cells from the extranuclear regions of the cell to the cell nucleus. The method comprises administering a polyamide and a molecular trafficking compound to the eukaryotic cells wherein the compound is selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof. The resulting effect of the molecular trafficking compound on the eukaryotic cell preferably results in a decrease in polyamide efflux, increase in polyamide influx, decrease of vesicular accumulation of polyamides, or a combination thereof.

In another embodiment, the present invention may be used as a method of modulating the expression of a gene in eukaryotic cell cultures or eukaryotic organisms. The method comprises administering a polyamide and a molecular trafficking compound to a eukaryotic cell cultures or eukaryotic organisms. The molecular trafficking compound acts on eukaryotic cells to favor the accumulation of polyamides in the nucleus thereby promoting or enhancing the effectiveness of the polyamides to bind to the target sites on the DNA. Once bound, the polyamides may enhance or inhibit gene expression. Preferably, the present invention may be used as a method of administering a molecular trafficking compound to promote the effectiveness of polyamide gene regulation treatment in mammalian cell cultures and mammalian organisms.

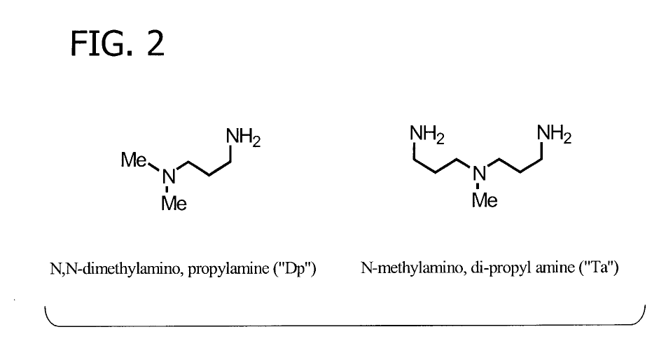

In another embodiment, the present invention relates to methods of modulating the cellular uptake and distribution of polyamide compounds within a eukaryotic cell by modifying the nature of the polyamide compound. Traditionally, polyamides are synthesized in a process that results in the polyamide compound having a weakly basic amine at the amino end (amino tail) of the polyamide chain giving these molecules weakly basic properties. The most common amine tail utilized is the N,N-dimethylaminopropyl group (“Dp”). Another amine tail that may be attached to a polyamide is that of N-methylamino, di-propylamine (“Ta”). See FIG. 2. The polyamide amine tails are the result of the solid phase synthesis of polyamides wherein the polyamides are cleaved from the solid support with by aminolysis with either Dp or Ta.

A weak base amine may also be incorporated at other positions in the polyamide structure, most notably on the hairpin corner. Accumulation of weakly basic drugs in acidic vesicles presumably occurs because these molecules are easily deprotonated at the near neutral pH of the cytoplasm. Once deprotonated, the molecule can pass through membranes. However, once the molecule passes into the lumen of a highly acidic vesicle, the molecule become protonated, and is unable to thereafter pass back across the membrane and into the cellular cytoplasm.

It has been surprisingly discovered that addition of a negatively charged acidic moiety to the polyamide via the amine tail, blocks vesicular accumulation and allows the polyamide to accumulate in the cell nucleus. By attaching the moiety to the amine tail, the attached moiety only mildly affects the binding affinity of the polyamide for DNA. In addition, in contrast to the traditional weakly basic polyamides that at physiological pH accumulate in acidic vesicles, negatively charged polyamides can be directly used to control gene expression in mammalian cells without further intervention (i.e., liposome delivery, lysosome disrupting agents) since they avoid lysosomal accumulation and instead accumulate in the nucleus where they can bind to the cellular DNA.

Negatively charged or acidic moieties that may be attached to polyamides to block their vesicular accumulation and promote accumulation in the cell nucleus include mildly acidic moieties. Examples of acidic moieties include, but are not limited to, fluorescein (fluorescein-5-isothiocyanate or FITC), phenol, carboxylic acid, HSO₃, and HₙPO₄ wherein n=1 to 3. The acidic moieties may be attached to polyamides by reacting the primary amine moieties on the amine tail of the polyamide with compounds containing the acidic moieties. Compounds containing acidic moieties include, for example, acryl, aromatic, alkyl, allyl, polyester compounds and the like.

In a preferred embodiment, a polyamide is synthesized wherein the polyamide contains a N-methylamino, di-propylamine (“Ta”) tail. See FIG. 2. The polyamide is reacted with fluorescein-5-isothiocyanate to attach fluorescein to the Ta tail.

In another embodiment, the present invention may be used as a method to modulate the distribution of a polyamide in a eukaryotic cell. The method comprises a administering a polyamide that contains a negatively-charged or acidic moiety and a molecular trafficking compound.

In a further embodiment, the present invention may be used as a composition to modulate the distribution of a polyamide in a eukaryotic cell. The composition comprises a polyamide and molecular trafficking compound. Compositions compising polyamides having negatively charged or acidic moieties and a molecular trafficking compound may also be administered to further promote the uptake and localization of the polyamide in the nucleus of a cell.

Molecular Trafficking Compound Dosing Regimen

The aforementioned molecular trafficking compounds may be administered in pharmaceutically acceptable concentrations to the cells or organisms possessing the target DNA according to methods known in the art. The molecular trafficking compound and the polyamide may be administered, separately, simultaneously, or sequentially to the cells or organisms. The route of administeration of the molecular trafficking compound may be administered orally, intravenously, intraperitoneally, subcutaneously, transdermally, and the like.

The dosing regimen of molecular trafficking compounds in the present invention is selected in accordance with a variety of factors. These factors include the selected molecular trafficking compound, the type, age, weight, sex, diet, and medical condition of the patient, the type and severity of the condition being treated with polyamide therapy, the target cell type being treated with polyamide therapy, the route of administration, pharmacological considerations such as the activity, efficacy, pharmacokinetics and toxicology profiles of the particular inhibitors employed, whether a drug delivery system is utilized, and whether the inhibitors are administered with other ingredients. Thus, the dosage regimen actually employed may vary widely and therefore deviate from the preferred dosage regimen set forth below.

Dosing may be with a regimen calling for a single daily dose, for multiple, spaced doses throughout the day, for a single dose every other day, for a single dose every several days, or other appropriate regimens.

A daily dose administered to a subject of about 0.001 to 30 mg/kg body weight, or between about 0.005 and about 20 mg/kg body weight, or between about 0.01 and about 15 mg/kg body weight, or between about 0.05 and about 10 mg/kg body weight, or between about 0.1 to 5 mg/kg body weight, may be appropriate. The amount of molecular trafficking compound that is daily administered to a human subject typically will range from about 0.1 to 2000 mg, or from about 1 to 1000 mg, or from about 5 to 800 mg, or from about 10 to 500 mg. The daily dose can be administered in one or more doses per day.

Where the molecular trafficking compound is verapamil, the oral daily dose administered typically is between about 40 mg to about 480 mg. Preferably, the oral daily dose is between about 120 mg to about 360 mg, more preferably between about 120 mg to about 240 mg. Illustrative oral daily doses of verapamil include, for example, 40, 80, 120, 240, 360 or 480 mg of verapamil.

Combinations of one or more molecular trafficking compounds may be administered to facilitate the uptake of polyamides within target cells. Selection of multiple molecular trafficking compounds is dependent upon factors such as target cell type, compatibility or contraindication of combining specific molecular trafficking compounds, and the like.

Polyamide Dosing Regimen

Polyamides may be administered in pharmaceutically acceptable concentrations to the cells or organisms possessing the target DNA according to methods known in the art. The route of administeration of the molecular trafficking compound may be administered orally, intravenously, intraperitoneally, subcutaneously, transdermally, and the like.

The polyamides may be administered generally to an organism through oral or parenteral routes. The polyamide may also be administered by injection or catheter to localize the polyamides to specific organs or tissues containing the target cells to be treated by polyamide therapy. The polyamides may be prepared in physiologically acceptable media in an appropriate form for the route of administration. Polyamide compositions may be prepared as powders, solutions, and dispersions in media for both oral and parenteral routes of administration.

The polyamides should be administered at a dosage that provides a polyamide concentration of about 1 nM to about 1 mM in the intracellular or extracellular location of the target cells. Preferably the polyamides should be provided at a dosage that provides a polyamide concentration of about 1 nM to about 100 μM in the intracellular or extracellular location of the target cells, more preferably between about 10 nm to 10 μM. In order to attain a desired concentration of polyamides inside the cell, the concentration of polyamides outside the cell in the extracellular sera should be approximately 2 to 1000 times greater in concentration.

The molecular trafficking compounds and polyamides may also be administered in combination with one or more additional therapeutic agents. Depending on the condition being treated, the combination therapy may also include antibiotics, vaccines, cytokines, anti-inflammatory drugs, and the like.

### EXAMPLE 1

### Preparation of Experimental Materials

Synthesis of Fluorescent-Labeled Polyamides

The polyamides Im-Py-Py-Py-γ-Py-Py-Py-Py-β-Ta and Im-Im-Py-Py-γ-Py-Py-Py-Py-β-Ta were prepared by the solid phase synthesis method as described by Baird, E.; Dervan, P. J. Am. Chem. Soc. 1996, 118, 6141.

Labeling of Polyamides with Fluorescent Probes

The polyamides were labeled with BODIPY-FL-X, SE and FITC respectively using standard labeling conditions.

FITC Labeling Method I

12 μl of diisopropylethylamine was added to a solution of 20.07 mg of tetra-trifluoroacetic acid salt of Im-Py-Py-Py-γ-Im-Py-Py-Py-β-Ta in 2 ml of anhydrous DMF (wherein “Ta”=NHCH₂CH₂CH₂N(Me)CH₂CH₂CH₂NH₂). An anhydrous DMF solution (2 ml) of fluorescein-5-isothiocyante (6.14 mg) (“FITC”) was added and the mixture stirred overnight at room temperature. The product was isolated via reverse phase chromatography using a methanol/water gradient. Lyophilization from a t-butanol/water mixture gave 17.2 mg of Im-Py-Py-Py-γ-Im-Py-Py-Py-β-Ta-FITC (74% yield). Mass spec: M+H⁺ (m/z=1655) and M+2H⁺ (m/z=828) observed.

FITC Labeling Method II

Im-Py-Py-Py-γ-Im-Py-Py-Py-β-Ta (5.58 mg) was placed in an oven-dried vial under N₂ gas and 0.25 ml of dry DMSO was added. After the solids dissolved, DIEA (10 μl) was added, followed by addition of fluorescein-5-isothiocyante (1.49 mg) solution in dry DMSO (300 μl). The reaction mixture was stirred at room temperature overnight in the dark. The product was isolated via preparative HPLC. Lyophilization from a t-butanol/water mixture gave 17.2 mg of Im-Py-Py-Py-γ-Im-Py-Py-Py-β-Ta-FITC (86.4% yield). Mass spec: M+H⁺ (m/z=1899) and M+2H⁺ (m/z=950) observed.

Cell Culture

Previously established low passage number human colon cancer HCT116 cells were cultured as monolayers and maintained at 37° C. in a humidified incubator with 5% CO₂ in buffered medium consisting of RPMI 1640 (GIBCO, Life Technologies, Rockville, Md.) supplemented with 2 mM L-glutamine, 10% fetal bovine serum (GIBCO), 100 Units/ml Penicillin-Streptomycin and 25 μg/ml Gentamicin. Previously established low passage number human rheumatoid synovial fibroblasts (RSFs) were cultured in DMEM (GIBCO 11995-040) supplemented with 15% FBS, 1% glutamine, and 50 g/ml Gentamycin.

Polyamide and Drug Treatment of Cell for Microscopy

HCT116 cells or RSF cells (8×10⁵) were plated on 25-mm round glass coverslips in 30-mm wells and incubated for 24 hr to allow cells to adhere. Fluorescent polyamides were freshly prepared in DMSO to 10 mM and then diluted to 1 mM with distilled water. The freshly prepared polyamide solution was then added pre-warmed cell culture media to a final concentration 10 μM polyamide, 0.1% DMSO. Cell culture media was removed from each well and replaced with the fresh polyamide-containing media and cells were incubated for an additional 16 hr at 37° C. in a humidified incubator with 5% CO₂. Where applicable, cells were also pretreated with one of the MDR inhibitors, verapamil, bepridil, cyclosporin A, or ketoconazole, at a concentration of 5 to 100 μM for 30 min before the addition of the fluorescent polyamides. After 30 min the media was removed and replaced with fresh polyamide-containing media supplemented with verapamil, bepridil, cyclosporin A, or ketoconazole (corresponding to pretreatment) and cells were incubated for 16 hr as above.

Organelle-Specific Fluorescent Probes

Where applicable, after the 16 hr incubation in the presence of polyamide and/or MDR inhibitor either MITOTRACKER Red CM-H₂XRos (Molecular Probes, Eugene, Oreg.), LYSOTRACKER Red DND-99 (Molecular Probes), or BODIPY® TR ceramide (Molecular Probes) was added directly to the cell culture for 15 min to 1 hr as recommended by the supplier. Just prior to examination 4,6-diamidino-2-phenylindole, dihydrochloride (DAPI) was added to cell cultures to 300 nM and samples were incubated for at least 5 min at room temperature. The coverslips were rinsed several times in PBS and wet mounted.

Fluorescence Microscopy

Live wet mounted cells were examined and photographed using an Olympus AX70 Microscope equipped with fluorescence optics and a Sony 3CCD color video camera. DAPI was detected using a bandpass 405±20 nm excitation filter, a 420 nm dichroic beam splitter, and a ≧450 emission filter (DAPI filter set). BODIPY and fluorescein conjugated polyamides were selectively detected using a bandpass 485±11 nm excitation filter, a 505 nm dichroic beam splitter, and a 530±15 nm emission filter (fluorescein filter set). Organelle-specific probes were detected using a 546+/−5 nm excitation filter, a 570 nm dichroic beam splitter and a 590 nm longpass emission filter (Rhodamine filter set).

### EXAMPLE 2

### Cellular Uptake of Polyamides

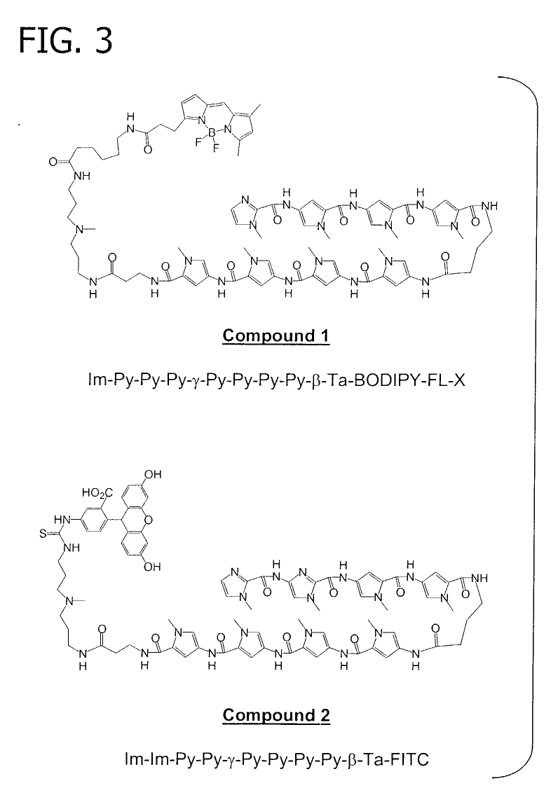

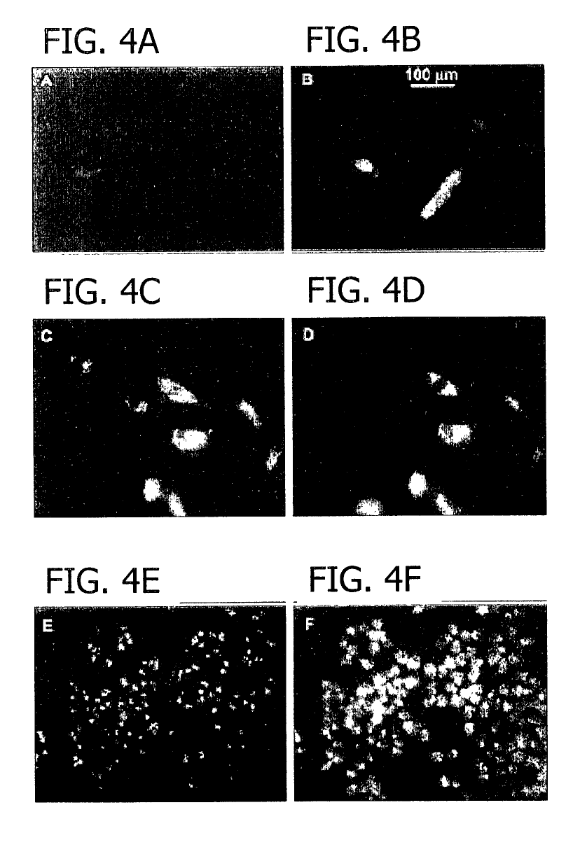

To examine uptake and intracellular distribution, polyamides were labeled with fluorescent probes (FIG. 3, compounds Compound 1 and Compound 2, BODIPY and fluorescein, respectively), cultured cells were treated with these fluorescent-polyamides, and the intracellular distribution was determined by fluorescence microscopy. FIG. 4 shows the fluorescence staining pattern of HCT116 human colon cancer cells and human rheumatoid synovial fibroblasts (RSF) treated with 10 μM Compound 1 overnight and counterstained with DAPI just prior to examination. In treated cells, fluorescence from Compound 1 showed a punctuated cytoplasmic pattern (FIGS. 4C & 4E) that did not overlap the DAPI stained nuclear DNA (FIGS. 4D & 4F). FIG. 4A shows that autofluorescence was undetected when untreated cells were examined using the BODIPY/FITC filter set (FIG. 4A synovial fibroblasts, HCT116 not shown) demonstrating that the fluorescence was due to Compound 1 and that Compound 1 entered the cells and accumulated in cytoplasmic compartments, not the nucleus. Similar punctuated cytoplasmic fluorescence was observed in HepG2 hepatocytes and RAW macrophage cells (data not shown). In addition, a similar intracellular distribution has previously been reported for a fluorescence DNA binding polyamide in SKOV-3 cells.

### EXAMPLE 3

### BODIPY-Labeled Polyamides Localize in Acidic Cytoplasmic Vesicles

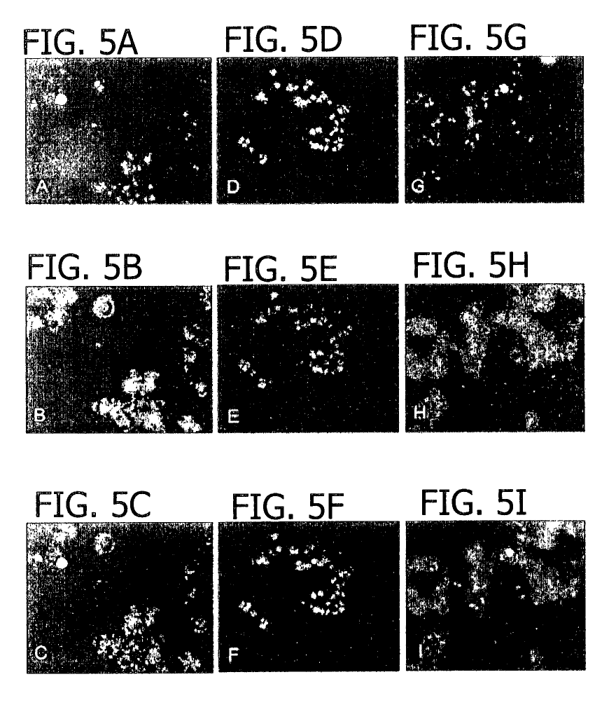

The distribution of Compound 1 in HCT116 and RSF cells suggested that Compound 1 is trafficked to a specific compartment within the cytoplasm. To determine which cytoplasmic compartment sequestered Compound 1, dual-staining co-localization studies were performed with Compound 1 and organelle-specific, red fluorescent probes. As shown in FIGS. 5A, 5D, and 5G, fluorescence from Compound 1 (green) was detected in cytoplasmic granules as above, which 1) did not overlap with MitoTracker fluorescence (FIG. 5B, overlay 5C), 2) showed partial co-localization with the golgi-specific probe fluorescence (FIG. 5H, overlay 5I), and 3) completely co-localized with LysoTracker fluorescence (FIG. 5E, overlay 5F). Therefore, Compound 1 clearly did not accumulate in mitochondria as previously reported, but rather accumulated in lysosomes and a portion of the golgi apparatus. The partial overlap of fluorescence from Compound 1 with the golgi-specific probe and complete overlap with LysoTracker is consistent with the fact that LysoTracker is specific for acidic organelles, and stains not only the lysosomes but also the trans-golgi. That Compound 1 has a weakly basic cationic nature is consistent with its accumulation in acidic organelles. It is well documented that weakly basic cations with pKₐs near neutral freely penetrate membranes, but once inside acidic organelles, become protonated and are thereby trapped in those compartments. This accumulation in acidic organelles, in turn, can prevent or reduce nuclear accumulation of a nuclear-targeted basic drug such as daunorubicin and doxorubicin, and likely accounts for the absence of detectable Compound 1 in the nucleus of RSF and HCT116 cells.

### EXAMPLE 4

### Verapamil Induces Nuclear Localization and Accumulation of Polyamides in RASFs but not HCT116 Cells

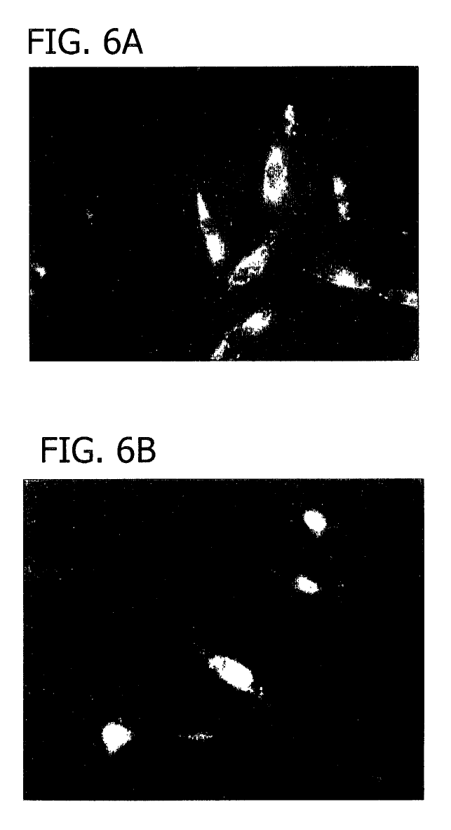

Sequestration of drugs in acidic vesicles has been described as one of the major mechanism responsible for multidrug-resistance. While not an absolute, the ability of a cell to sequester drugs into vesicles is a phenotype frequently co-expressed with the cells ability or increased capacity to efflux drugs or substrates via the plasma membrane transporter, p-glycoprotein (P-gp). Because of this, we attempted to induce nuclear accumulation of Compound 1 using P-gp inhibitors including verapamil, bepridil, cyclosporin A, and ketoconazole. RSF and HCT116 cells were treated with Compound 1 as before, but in the presence of the P-gp inhibitors. None of these showed any observable ability to enhance nuclear accumulation of Compound 1 or have any observable effect on the intracellular distribution of Compound 1 in HCT116 (data not shown), indicating that Compound 1 is not likely a substrate for p-glycoprotein-mediated efflux in these cells. Bepridil, cyclosporin A, and ketoconazole also had no effect on the intracellular distribution of Compound 1 in RSF cells (data not shown). However, verapamil treatment induced a dramatic reduction in cytoplasmic fluorescence accompanied by a re-distribution of Compound 1 to the nucleus in RSF (FIGS. 6B & 6C). The dramatic reduction in cytoplasmic fluorescence indicates that verapamil blocked vesicular sequestration of Compound 1 and suggests that vesicular sequestration of Compound 1 in untreated cells is the main reason that Compound 1 does not reach its intended intracellular target (the nucleus) in untreated cells. Importantly, we infer that the nuclear envelope does not act as a barrier to Compound 1. The mechanism by which verapamil blocks vesicular sequestration of Compound 1 in RSF cells is unclear. However, verapamil not only inhibits p-glycoprotein-mediated drug efflux, but also affects intracellular Ca+ concentration which, in turn, influences a number of intracellular events including vesicular trafficking. In addition, verapamil, itself, is a cationic, weak base and has been shown to accumulation in acidic vesicles in response to the proton electrochemical gradient across the vesicular membrane. It is therefore likely that verapamil-induced nuclear accumulation of Compound 1 results from disruption in the trafficking or the general homeostasis of acidic vesicles. Such mechanisms have previously been shown to increase the sensitivity of drug resistant cells to nuclear-targeted weakly basic cationic drugs.

### EXAMPLE 5

### Fluorescein-Labeled Polyamides Accumulate in the Nucleus of HCT116 Cells

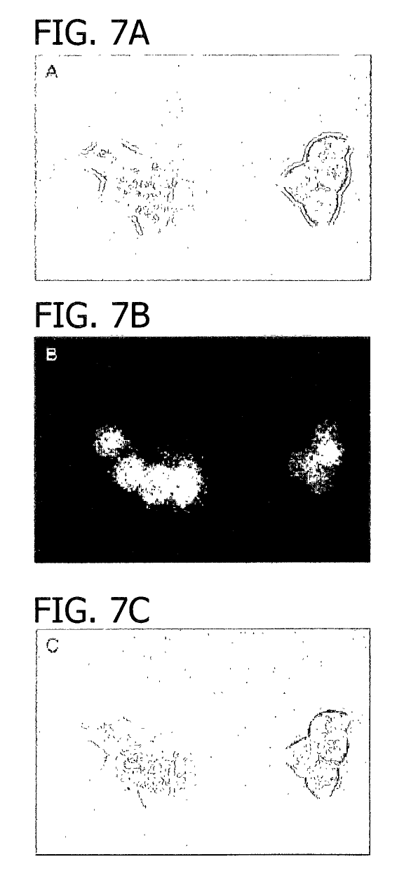

Based on the results above, the exclusion of Compound 1 from the nucleus of HCT116 cells and RSF (in the absence of verapamil) appears to be due to vesicular sequestration and not inability to cross the nuclear envelope. In general, it is the neutralization of the charge on weakly basic cationic drugs that allow them to cross membranes, including that of the acid vesicles. In attempt to block diffusion of Compound 2 through the vesicular membranes and, therefore, to block vesicular accumulation of Compound 2, we modified a related polyamide such that it contained an acid anionic moiety (compound 2) that was not susceptible to neutralization at physiological or near physiological pH. HCT116 cells treated with Compound 2 are shown in FIG. 7. Fluorescent-filled vesicles are absent (FIG. 7B), while nuclei are brightly stained (FIG. 7C). We conclude that the additional anionic moiety did block Compound 2 from crossing the membranes of acidic vesicles, thereby blocking vesicular accumulation. Presumably, with this pathway blocked and because of the permeable nature of the nuclear envelope, Compound 2 entered the nucleus and accumulated therein due to its high affinity for DNA. Again, this demonstrates that the nuclear envelope does not act as a barrier to polyamides.

The results reported here show that vesicular accumulation of a polyamide can be inhibited by agents that disrupt the acidic vesicle homeostasis or by modifying the charge of the polyamide. When vesicular sequestration is inhibited, polyamides are free to accumulate in the nucleus. Since the nuclear DNA is the polyamide target, polyamides may indeed be useful molecules for regulating gene expression in mammalian cells by using one of these two strategies.

In view of the above, it will be seen that the several objects of the invention are achieved.

As various changes could be made in the above compositions and processes without departing from the scope of the invention, it is intended that all matter contained in the above description be interpreted as illustrative and not in a limiting sense.

What is claimed is:

1. A method for modulating the distribution of a polyamide within eukaryotic cells, the method comprising:
administering to the eukaryotic cells a molecular trafficking compound selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof, and
administering the polyamide to the eukaryotic cells.

2. The method of claim 1, wherein the eukaryotic cell is a mammalian cell.

3. A method of claim 1, wherein the polyamide is distributed from the extranuclear region of the cell to the nucleus of the cell.

4. The method of claim 1, further comprising administering, simultaneously or sequentially, the molecular trafficking compound and the polyamide.

5. The method of claim 4, wherein the molecular trafficking compound and the polyamide are simultaneously administered as a mixture.

6. The method of claim 1, wherein more than one polyamide and one molecular trafficking compound are administered.

7. The method of claim 1, wherein one polyamide and more than one molecular trafficking compound are administered.

8. The method of claim 1, wherein more than one polyamide and more than one molecular trafficking compound are administered.

9. The method of claim 1, wherein the molecular trafficking compound is a P-glycoprotein inhibitor.

10. The method of claim 1, wherein the molecular trafficking compound is an ATPase affecting chemical.

11. The method of claim 1, wherein the molecular trafficking compound is a pH or proton gradient disrupter.

12. The method of claim 1, wherein the molecular trafficking compound is a calcium channel blocker.

13. The method of claim 1, wherein the molecular trafficking compound is an ATP depleting chemical.

14. The method of claim 1, wherein the molecular trafficking compound is a sodium/potassium channel blocker.

15. The method of claim 1, wherein the molecular trafficking compound is a MRP inhibitor.

16. The method of claim 1, wherein the molecular trafficking compound is a protein kinase inhibitor.

17. The method of claim 1, wherein the molecular trafficking compound is a Multidrug Resistance Compound.

18. A method for modulating the expression of a gene in cells of a eukaryotic organism comprising:
administering a molecular trafficking compound selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof, and
administering one or more polyamides to the eukaryotic cells.

19. The method of claim 18, wherein the eukaryotic organism is a mammal.

20. The method of claim 18, wherein the polyamide is a polyamide that contains an acidic moiety.

21. A method for modulating the distribution of a polyamide within eukaryotic cells, the method comprising, conjugating an acidic moiety to the polyamide to produce a modified polyamide and administering the modified polyamide to eukaryotic cells.

22. The method of 21 wherein the acidic moiety is selected from the group consisting of fluorescien, phenol, carboxylic acid, HSO₃, and HₙPO₄ wherein n=1 to 3.

23. The method of claim 21 wherein the the acidic moiety is conjugated to an amine present on the polyamide.

24. The method of claim 23, wherein the amine is located on an amine tail of the polyamide.

25. The method of claim 24, wherein the amine is a primary amine.

26. The method of claim 24 wherein the amine tail of the polyamide comprises N-methylamino, di-propylamine.

27. The method of claim 21 wherein the acidic moiety is conjugated to the polyamide with a linking group wherein the linking group is selected from the group consisting of acryl, aromatic, alkyl, allyl, and polyester groups.

28. A composition for modulating the expression of a gene in a eukaryotic cell, the composition comprising a polyamide and a molecular trafficking compound wherein the molecular trafficking compound is selected from the group consisting of P-glycoprotein inhibitors, ATPase affecting chemicals, pH or proton gradient disrupters, calcium channel blockers, ATP depleting chemicals, sodium/potassium channel blockers, MRP inhibitors, protein kinase inhibitors, Multidrug Resistance Compounds and combinations thereof.

29. The composition of claim 28, wherein the composition comprises more than one polyamide.

30. The composition of claim 28, wherein the polyamide is a polyamide that contains an acidic moiety.
